(19) United States
(12) Patent Application Publication
(10) Pub. No.: US 2009/0291887 A1
(43) Pub. Date: Nov. 26, 2009

(54) Proteins of the SDF-1-Family for the Manufacturing of a Medicament

(76) Inventors: Hans-Werner Müller, Dusseldorf (DE); Herbert Tröscher, Heiligenhaus (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/307,614

(22) PCT Filed: Jul. 6, 2007
(86) PCT No.: PCT/EP2007/056906

§ 371 Date: May 7, 2009

(30) Foreign Application Priority Data
Jul. 7, 2006 (EP) 06116845.6

(51) Int. Cl. A61K 38/19; A61P 17/02

(52) U.S. Cl. 514/12

(57) ABSTRACT

Use of a protein of the SDF-1-family for the manufacturing of a medicament for the improvement of the plasticity and/or regeneration of axons upon their lesion.

The present invention pertains to the use of a protein of the SDF-1-family for the manufacturing of a medicament to improve the plasticity, sprouting and/or regeneration of axons upon their lesion.

Injuries of nerves often lead to non-reparable damages in particular when axons of the nerve cells are cut by said injuries. Due to the limited regeneration of the axons permanent damages of the patients remain. Among others it is an object of the invention to provide means which effect an improvement of regeneration of axons upon their lesions.

This aim is accomplished by the use of a protein of the SDF-1-family for manufacturing of a medicament for the improvement of plasticity and/or sprouting and/or regeneration of axons upon their lesion.

Repair of traumatic and ischemic lesions of the spinal cord or brain, such as injuries, caused e.g. by accidents but also stroke and other ischemic insults to the nervous system, requires growth of neurites, either as compensatory sprouting of spared fibres that have not been affected by the trauma, or as true regenerative growth of lesioned axons (Maier and Schwab, 2006).

The term neuroplasticity is defined as the brain's ability to reorganize itself by forming new neural connections throughout life. Neuroplasticity allows the neurons (nerve cells) in the brain to compensate for injury and disease and to adjust their activities in response to new situations or to changes in their environment.

Brain reorganization takes place by mechanisms such as “axonal sprouting” in which undamaged axons grow new nerve endings to reconnect neurons whose links were injured or severed. Undamaged axons can also sprout nerve endings and connect with other undamaged nerve cells, forming new neural pathways to accomplish a needed function.

For example, if one hemisphere of the brain is damaged, the intact hemisphere may take over some of its functions. The brain compensates for damage in effect by reorganizing and forming new connections between intact neurons. In order to reconnect, the neurons need to be stimulated through activity. (Webster's New World Medical Dictionary)

Both types of neurite growth are abundant following injuries to the newborn central nervous system (CNS). Until this time period axons are still plastic. There are no proteins in the surrounding that impede sprouting, growth and regeneration. This window of opportunity closes, however, as CNS development ends within a few weeks postnatally in rodents, and in a few months in humans (Chen et al., 2002). Simultaneously, the cellular composition of the CNS changes dramatically by the differentiation of oligodendrocytes and the myelination of axons (Kapfhammer and Schwab, 1994). In this point of development the natural plasticity and therefore the ability to compensate for neuronal loss begins to be restricted (Maier and Schwab, 2006).

Enhancement of plasticity by induction of compensatory sprouting has been shown to compensate for formerly lost function in spinal cord injury models, (Galtrey and Fawcett, 2007), (Galtrey et al., 2007), as well as in stroke models (Seymour et al., 2005; Markus et al., 2005).

In this application we introduce a new molecule family, the chemokine family of stromal cell derived factor one, SDF-1, that has been shown to overcome inhibitory influences by induction of sprouting as is shown by the presented experimental data. These findings lead to the development of a medicament for the improvement of plasticity and/or sprouting and/or regeneration of axons upon their lesion. Such a medicament can be used for the treatment of traumatic, ischemic and idiopathic insults to the nervous system, as well as for the treatment of neurodegenerative diseases, such als Alzheimer's Disease, Parkinson's Disease and Multiple Sclerosis. FIG. 1 is showing the growth of DRG-neurons on laminin.

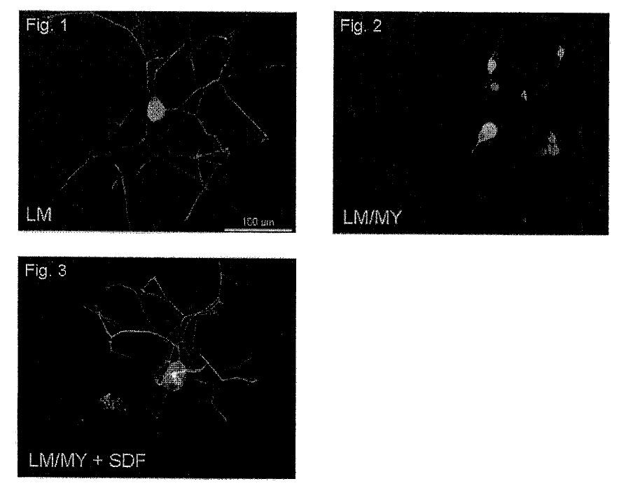

FIG. 2 is showing DRG-neurons on laminin in the presence of myelin.

FIG. 3 is showing the growth of DRG-neurons on laminin/myelin in presence of SDF-1 Alpha (200 ng/ml).

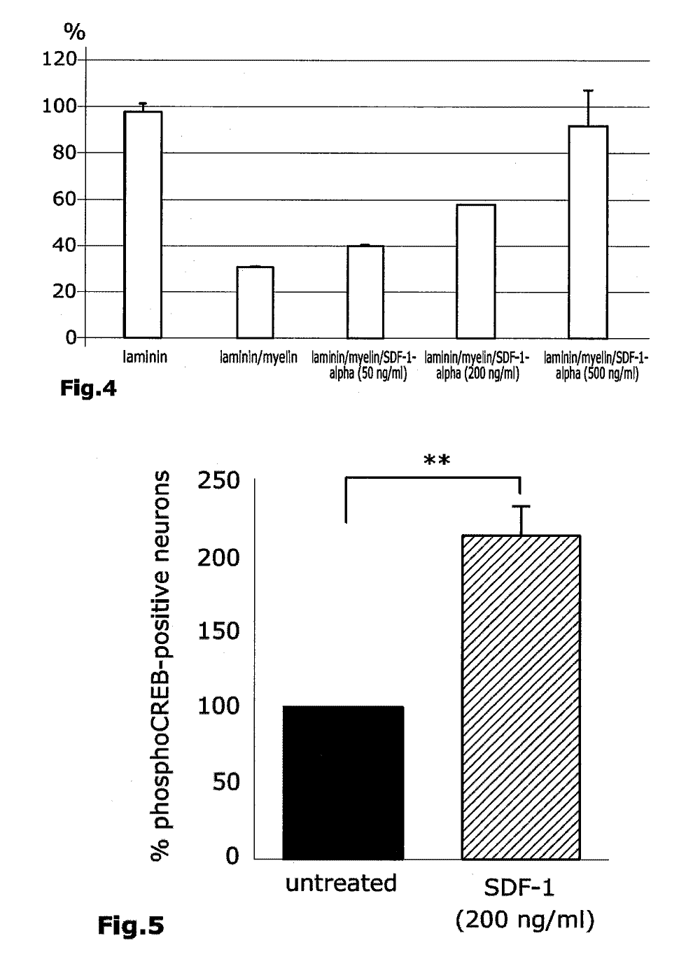

FIG. 4 is showing a quantitative evaluation proving the induction of sprouting on the inhibitory myelin-substrate by SDF-1.

FIG. 5 shows the quantification of phospho-CREB immunopositive neurons after SDF-treatment.

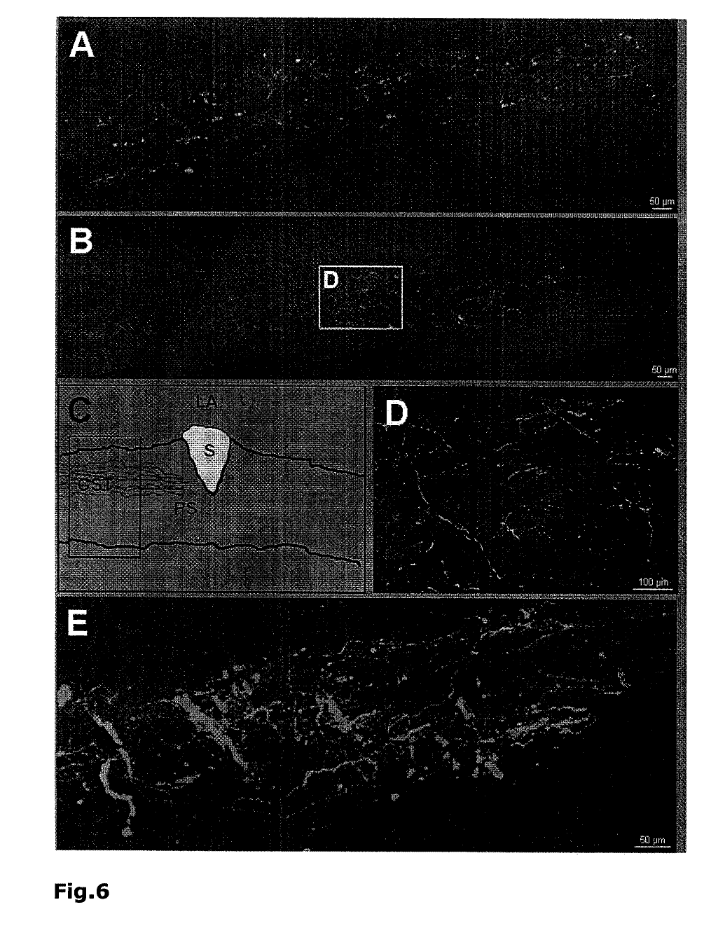

FIG. 6 shows the induction of axonal sprouting by administration of SDF in vivo, in a rat model of spinal cord transection.

Typically, the lesions can be induced by traumatic injuries, inflammatory, ischemic, and/or neuro degenerative processes.

Besides the native protein of the SDF-1-family other proteins of the SDF-1-family are to be taken into account, which proteins are at least 80% homologous with the naturally occurring SDF-1-protein. In particular proteins of the SDF-1-family are used which are at least 90% homologous more particular at least 95% homologous to the native protein. The term homology is well known to the skilled person and means, according to accepted understanding, identity of the amino acid sequence of a given protein.

According to the invention SDF-1-proteins can be used, which are selected from the group consisting of SDF-1 Alpha, SDF-1 Beta, SDF-1Gamma, SDF-1 Delta, SDF-1 Epsilon und SDF-1 Phi. Also variants, mutants, and/or fragments and chimeric molecules that are derived from SDF-aminoacid sequence parts exhibiting the biological effect of the SDF-1-protein. According to the invention the term “variants” mean proteins which are derived from SDF-1 proteins and may be generated by way of e.g. splicing, mutation, substitution of amino acids or proteolytic cleavage but have remained substantially the same or equivalent biologial activity of the starting protein SDF-1. Likewise derivatives of the afore mentioned proteins can be employed. According to the invention the term “derivative” means such proteins which are functionalized by functional groups of the peptide side chain or are chemically modified. By way of example are mentioned phosphorylated, amidated, sulphated or glycosylated proteins.

The skilled person can easily find the appropriate dosage of the protein of the SDF-1-family. Typically, the dosage is in the range of from about 1 ng to 1 mg per kilogram body weight. The skilled person can also easily determine the galenic formulation depending on the manner of application of the medicament. Solutions having physiological consistence are preferred by intraveneous, intrathecal, intraventricular or intramedular administration.

The use of the invention of the SDF-1-protein provides a process for the improvement of plasticity and/or regeneration of axons wherein a protein of the SDF-1-family is administered to a patient in need thereof. The proteins which can be employed in the process of the invention are equivalent to those described hereinabove. According to the process of the invention the protein of the SDF-1-family is administered locally, intramedularly, intraventricularly, intrathecally, or intravenously.

Subject matter of the invention is also a process for the improvement of plasticity, sprouting and/or regeneration of axons wherein a protein of the SDF-1-family, the use of which is claimed is administered to a patient in need thereof. In such process the protein of the SDF-1-family is administered to the patient in a suitable physiologically acceptable galenic formulation in amounts of from about 1 ng to about 1 mg.

The following examples show unexpected novel poperties of SDF-1 in vitro which contribute to an improvement of axonal plasticity and regeneration in vivo.

On cultured neurons from the dorsal root ganglia (DLR) of the rat, whose central axons are responsible for the sensory input into the spinal chord and run towards the head in the dorsal funiculus of the spinal chord, it is shown that myelin inhibits the outgrowth of the neurons. The myelin-associated inhibition of axon growth is attributed to inhibitor molecules such as NOGO and MAG and is very well substantiated in the literature by the working groups of Schwab (Schnell, L. and Schwab, M., Nature, 1990, 343: 269-72), McKerracher (Li et al., J. Neurosci. Res. 1996, 46: 404-414) and Filbin (Filbin, M. T., Nat Rev Neurosci. 2003, 4: 703-13).

The following examples show that this myelin inhibition by SDF-1 can be significantly suppressed.

### EXAMPLES

### Methods:

### Myelin-Inhibition Assay:

Spinal neurons from the dorsal root ganglia (DRG) of young rats (day 6 postnatal) were plated in parallel on culture substrates coated with either poly-D-lysine (PDL)/laminin (13 μg/ml) or PDL/laminin (13 μg/ml)+myelin (200 ng/culture). The myelin preparation has been obtained from rat brain by biochemical fractionation and employed as a suspension for coating the culture dishes.

### PhosphoCREB Assay:

Determination of Ser-133 phosphorylated CREB was performed on glass coverslips coated with PDL (1 mg/ml, Sigma) and laminin (13 μg/ml, Sigma). Dissociated DRGs were plated at a density of 5×104 cells/cm2 and incubated in DMEM containing 10% fetal bovine serum, nerve growth factor-2.5S, penicilline/streptomycine, and 5′-fluoro-2′-desoxyuridine for 24 h. Cells were stimulated by application of either 200 ng/ml SDF-1a/CXCL12 or 6 μM forskolin (Sigma), respectively, for 1-120 min. Samples were fixed with 40% PFA and stained for phophoCREB. Nuclei of P6 DRG neurons were stained with DAPI. For quantitative analysis of phosphoCREB-immunoreactivity, 30 pictures per coverslip were taken randomly at 20× magnification, and the number of neurons displaying phosphoCREB-positive nuclei as well as the total number of neurons were determined.

Spinal Cord Surgery and Application of SDF-1a/CXCL12:

Transection of the CST and dorsal columns was performed as described previously (Hermanns et al., 2001a). The dura was opened at the thoracic level, and the vertebrae Th8 and Th9 were removed. The dorsal CST, dorsal columns and central canal were transected using a Scouten wire knife (Bilaney, Germany). Following the surgery, a silicon tube catheter was placed in the subdural space at 1 mm caudal to the lesion, and was connected to a mini-osmotic pump (Alzet) containing 200 μl of either SDF-1a/CXCL12 (10 μM in PBS) or Tris buffer. After the pump was fixed in place, the dura and overlying tissue were sutured, and the animals were allowed to recover. Application of either SDF-1a/CXCL12 or Tris buffer was carried out at a flow rate of 1 μl/h over a time period of 7 days. One week after lesion, the pump was removed in a second surgery.

### Anterograde CST Axon Tracing and Immunohistochemistry:

Four weeks before killing, SDF-1a- and Tris buffer-infused rats received CST axon tracing as described previously (Klapka et al., 2005). A total volume of 2.3 μl biotinylated dextrane amine, BDA, (10%, Molecular Probes) was stereotactically injected into both hemispheres of the sensorimotor cortex. Tissue preparation for immunohistochemistry and axon tracing was performed as described previously (Hermanns and Müller, 2001). Briefly, animals were transcardially perfused with 40% PFA. The spinal cord was removed, postfixed in 4% PFA, and cryoprotected in sucrose (30%, Sigma) for 3-5 days. Spinal cord samples were shock frozen in methyl butane and cut into 20 μm thick sagittal cryostat slices. Sections were collected onto Histobond coated glass slides (Menzel, Germany). Immunostaining of spinal cord slices for BDA was performed as described previously (Klapka et al., 2005). Briefly, sections were washed in PBS followed by incubation with avidin-Oregon (1:1000, in PBS) at 4° C. over night. The next day, samples were washed in PBS prior to DAPI staining and mounting in Fluoromount-G (SouthernBiotech). Immunofluorescence was visualized and images were captured with a Nikon Diaphot 300 (Nikon) using NIS FreeWare 2.10.

FIG. 1 shows the pronounced axon growth of DRG neurons on PDL/laminin substrate. This massive growth of the axons is strongly inhibited by adding a myelin preparation from rat brain (FIG. 2). However, the myelin-associated inhibition of neurite growth can be essentially neutralized by adding SDF-1α (FIG. 3).

The images shown in FIGS. 1-3 were recorded by immunofluorescence microscopy with PAM (panaxonal marker/neurofilament) antibody, and the visualization was effected by fluorescent dye (Alexa).

FIG. 4 shows the quantitative evaluation relating to the dose-dependent neutralization of myelin inhibition by the chemokine SDF-1.

The myelin-induced inhibition of the neurite growth of DRG neurons in vitro can be neutralized completely by a concentration of 500 ng of SDF-1/ml.

FIG. 5 shows the quantification of phospho-CREB immunopositive neurons after SDF-treatment.

Application of SDF-1a/CXCL12 leads to Ser-133 phosphorylation of CREB in DRG neurons. Nuclei of untreated P6 DRG neurons generally show no pCREB-immunoreactivity (data not shown). Numbers of phosphoCREB-positive nuclei are low in untreated cultures in vitro. Treatment of neurons with SDF-1a/CXCL12 at a concentration of 200 ng/ml results in a significantly increased proportion of nuclei displaying phosphoCREB-immunoreactivity (FIG. 5).

FIG. 6 shows the induction of axonal sprouting by administration of SDF in vivo, in a rat model of spinal cord transection.

SDF-1a induces sprouting in CST-lesioned adult rats. Axonal growth is impaired following spinal cord transection of the CST. Sprouting of BDA-labelled axons within the proximal stump does occur only randomly in Tris buffer-treated control animals (A, C). Conversely, application of SDF-1a is followed by enhanced sprouting of CST axons and effects extensive branching of sprouting fibres (B, D). A considerable amount of sprouting is also observed after cAMP-treatment (E). CST, corticospinal tract; LA, lesion area; PS, proximal stump; S, scar. Two out of three animals displayed extensive sprouting following SDF-1a-treatment, whereas in none out of three Tris buffer-infused rats sprouting was observed within the proximal stump. Frame in (B) shows field in (D).

### REFERENCE LIST

1. Chen R, Cohen L G, Hallett M (2002) Nervous system reorganization following injury. Neuroscience 111: 761-773.

2. Galtrey C M, Asher R A, Nothias F, Fawcett J W (2007) Promoting plasticity in the spinal cord with chondroitinase improves functional recovery after peripheral nerve repair. Brain 130: 926-939.

3. Galtrey C M, Fawcett J W (2007) The role of chondroitin sulfate proteoglycans in regeneration and plasticity in the central nervous system. Brain Res Rev 54: 1-18.

4. Kapfhammer J P, Schwab M E (1994) Inverse patterns of myelination and GAP-43 expression in the adult CNS: neurite growth inhibitors as regulators of neuronal plasticity? J Comp Neurol 340: 194-206.

5. Maier I C, Schwab M E (2006) Sprouting, regeneration and circuit formation in the injured spinal cord: factors and activity. Philos Trans R Soc Lond B Biol Sci 361: 1611-1634.

6. Markus T M, Tsai S Y, Bollnow M R, Farrer R G, O'Brien T E, Kindler-Baumann D R, Rausch M, Rudin M, Wiessner C, Mir A K, Schwab M E, Kartje G L (2005) Recovery and brain reorganization after stroke in adult and aged rats. Ann Neurol 58: 950-953.

7. Seymour A B, Andrews E M, Tsai S Y, Markus T M, Bollnow M R, Brenneman M M, O'Brien T E, Castro A J, Schwab M E, Kartje G L (2005) Delayed treatment with monoclonal antibody IN-1 1 week after stroke results in recovery of function and corticorubral plasticity in adult rats. J Cereb Blood Flow Metab 25: 1366-1375.

1-9. (canceled)

10. A method for the improvement of the plasticity, sprouting and/or regeneration of axons upon their lesion, comprising administering a protein of the SDF-1-family, or variant, mutant, or fragment thereof having a biological effect of a protein of the SDF-1-family.

11. The method of claim 10, wherein the lesion is caused by traumatic injuries, inflammatory, ischemic, and/or neurodegenerative processes.

12. The method of claim 10, wherein the variant, mutant, or fragment having a biological effect of a protein of the SDF-1-family is at least 80% homologous to a protein of the SDF-1-family.

13. The method of claim 12, wherein the variant, mutant, or fragment having a biological effect of a protein of the SDF-1-family is at least 90% homologous to a protein of the SDF-1-family.

14. The method of claim 13, wherein the variant, mutant, or fragment having a biological effect of a protein of the SDF-1-family is at least 95% homologous to a protein of the SDF-1-family.

15. The method of claim 10, wherein the protein of the SDF-1-family, or variant, mutant, or fragment having a biological effect of a protein of the SDF-1-family, is selected from the group consisting of SDF-1 Alpha, SDF-1 Beta, SDF-1 Gamma, SDF-1 Delta, SDF-1 Epsilon, and SDF-1 Phi.

16. The method of claim 10, wherein the protein of the SDF-1-family, or variant, mutant, or fragment thereof having a biological effect of a protein of the SDF-1-family, is administered to a subject in need thereof at a dosage of from about 1 ng to about 1 mg per kg body weight.

17. The method of claim 10, wherein the protein of the SDF-1-family, or variant, mutant, or fragment thereof having a biological effect of a protein of the SDF-1-family, is administered to a subject in need thereof locally, intramedularly, intraventricularly, intrathecally, or intravenously.

18. The method of claim 10, wherein the protein of the SDF-1-family, or variant, mutant, or fragment thereof having a biological effect of a protein of the SDF-1-family, is administered to a subject in need thereof in a physiologically acceptable galenic formulation.
